(19) United States
(12) Patent Application Publication
(10) Pub. No.: US 2022/0265126 A1
(43) Pub. Date: Aug. 25, 2022

(54) OVERTUBE AND MEDICAL DEVICE USING THIS OVERTUBE

(71) Applicants: Christophe BASTID, Marseille (FR); Thierry MANOS, Marseille (FR)

(72) Inventors: Christophe BASTID, Marseille (FR); Thierry MANOS, Marseille (FR)

(21) Appl. No.: 17/629,006

(22) PCT Filed: Jul. 31, 2020
(86) PCT No.: PCT/FR2020/051409

§ 371 Date: Jan. 21, 2022

(30) Foreign Application Priority Data
Jul. 22, 2019 (FR) FR1908305

(51) Int. Cl. A61B 1/00; A61B 1/015; A61B 1/018

(52) U.S. Cl. CPC A61B 1/00135; A61B 1/015; A61B 1/018; A61B 2017/2927

(57) ABSTRACT

An overtube that includes a body, a first duct for housing a first insertion tube of a first endoscope, where the first duct is arranged longitudinally within the body and opens at the distal end, a second duct for housing a second insertion tube of a second endoscope, where the second duct is arranged longitudinally within the body and opens at the distal end, a first orifice for introducing the first insertion tube into the first duct, a second orifice for introducing the second insertion tube into the second duct, a third orifice for introducing a lubricant into the first duct and into the second duct, and where the first duct and the second duct communicate with each other via a communication passage, and the passage opens at the third orifice.

### TECHNICAL FIELD

The invention relates to an overtube and a medical device using the overtube.

The invention relates to the technical field of accessories for introducing endoscopes into a body cavity.

### BACKGROUND

Sleeve gastrectomy is an invasive bariatric surgery technique aimed at reducing the size of the stomach in order to allow weight loss. Endo-sleeves form integral part of bariatric endoscopy, and use an endoscopic technique that makes it possible to create folds in the gastric wall and to suture them in order to reduce the volume of the stomach (transoral gastric plication or transoral gastroplasty).

Because the gastric wall is relatively thick and hard, suturing the folds is a difficult and time-consuming task and requires specially dedicated equipment. Medical devices dedicated to such procedures have for example been described in patent documents US2016/0235400, US2007/0197862 or US2005/0251166. These medical devices use a single endoscope, often with two operator channels, allowing the passage of two surgical instruments: one that pinches the gastric wall and forms the fold, and another that sutures the fold.

However, these devices are complex to make and relatively expensive. As a result, they require significant financial investment from the user.

One aim of the invention is to remedy that state of affairs. Another aim of the invention is to propose an accessory with a simple and inexpensive design, which makes it possible to perform bariatric endoscopy very simply, for a lower cost.

### SUMMARY

The solution proposed by the invention is an overtube comprising:

a longitudinally extending flexible tubular body having a proximal portion and an open distal end,

a first duct designed to house a first tube for inserting a first endoscope into the body, with the possibility of longitudinal displacement of said first tube in said first duct and rotational movement of said first tube about its longitudinal axis, which first duct is arranged longitudinally inside said body and opens at the distal end of said body,

a second duct designed to house a second tube for inserting a second endoscope into the body, with the possibility of longitudinal displacement of said second tube in said second duct and rotational movement of said second tube about its longitudinal axis, which second duct is arranged longitudinally inside said body and opens at the distal end of said body,

a first orifice made at the proximal portion of the body for introducing the first insertion tube into the first duct,

a second orifice made at the proximal portion of the body for introducing the second insertion tube into the second duct,

a third orifice made at the proximal portion of the body for introducing lubricant into the first duct and into the second duct,

and wherein the first duct and the second duct communicate with each other over their entire length through a communication passage, which opens at the third orifice.

The overtube is an accessory generally used to insert a single endoscope insertion tube into a body cavity. The overtube inserted into the esophagus, through the patient's mouth, up to the stomach. The overtube according to the invention is now configured to receive two endoscopes. The user can therefore use this new overtube with two conventional endoscopes, each with specific instrumentation and an optical vision device. The user can thus introduce in the body cavity (in particular in the stomach), two distinct surgical instruments (one per endoscope) to carry out the surgical procedure, as each instrument can be observed along its own axis of view. The user no longer needs to purchase an expensive dual operator channel endoscope as described in the aforementioned prior art. Lubricant is introduced in the two ducts simply, from the third orifice, as the opening passage between the two ducts allows them to be lubricated simultaneously. The design of the overtube is particularly simple, making it inexpensive and easy to use.

Other advantageous characteristics of the invention are listed below. Each of these characteristics may be considered alone or in combination with the outstanding characteristics defined above. Where applicable, each of these characteristics contributes to the resolution of specific technical problems defined further in the description, to which the outstanding characteristics defined above do not necessarily contribute. Where applicable, these may be the subject of one or more divisional patent applications:

the first duct, the second duct and the communication passage are advantageously configured so that the first insertion tube of the first endoscope and the second insertion tube of the second endoscope do not touch each other when said tubes are housed in said ducts.

Advantageously, the communication passage connecting the first duct to the second duct is narrower than said ducts.

Advantageously, the body has a cross-section in the shape of an 8.

Advantageously, the body has a length ranging from 250 mm to 1500 mm.

Another aspect of the invention relates to a medical device comprising:

a first endoscope comprising:

a first flexible insertion tube having a first distal portion equipped with tip deflection, inside which is arranged a first operating channel for the insertion of a first surgical instrument (advantageously forceps with wide jaws), which first channel opens at a first distal end of said first tube,

a first optical viewing device located at the first distal end of the first insertion tube,

a first control handle connected to a proximal end of the first insertion tube,

a second endoscope comprising:

a second flexible insertion tube having a second distal portion equipped with tip deflection, inside which is arranged a second operating channel for inserting a second surgical instrument (advantageously a surgical stapling device, or a mounted clip), which second channel opens out at a second distal end of said second tube,

a second optical viewing device located at the second distal end of the second insertion tube,

a second control handle connected to a proximal end of the second insertion tube,

an overtube according to one of the foregoing characteristics, wherein:

the first insertion tube is housed in the first duct, with the first distal portion and the first distal end of said first tube protruding out of said first duct and the body through the distal end of said body,

the second insertion tube is housed in the second duct, with the second distal portion and the second distal end of said second tube protruding out of said second duct and the body through the distal end of said body,

### BRIEF DESCRIPTION OF THE FIGURES

Other advantages and characteristics of the invention will become clearer upon reading the description of the following preferred embodiment, by reference to the appended drawings, provided for guidance as non-limiting examples, wherein:

FIG. 1 is a perspective view of a conventional endoscope, used in the invention, as well as an enlargement of the head of said endoscope.

FIG. 2 is a drawing of an overtube according to the invention.

FIG. 3 is a cross-sectional view along A-A of the overtube of FIG. 2.

FIG. 4 is a perspective view showing design details of a medical device according to the invention.

FIG. 5 is a drawing of the placing of a medical device according to the invention in a patient's body.

FIG. 6 is a drawing of the formation of a fold in the gastric wall of a patient and suturing using the medical device according to the invention.

### DETAILED DESCRIPTION OF THE EMBODIMENTS

For clarity, certain terms used in the description and claims have been defined as follows:

“A and/or B” means: A alone or B alone or A+B.

unless otherwise specified, the use of ordinal adjectives “first”, “second”, etc., to describe an object simply indicates that various occurrences of similar objects are mentioned and does not imply that the objects thus described must be in any given sequence, whether in time, space, classification, or in any other way.

The medical device according to the invention comprises an overtube and two conventional endoscopes. It is particularly designed for bariatric surgery procedures, and more specifically bariatric endoscopy, but may be used for other endoscopic surgery procedures.

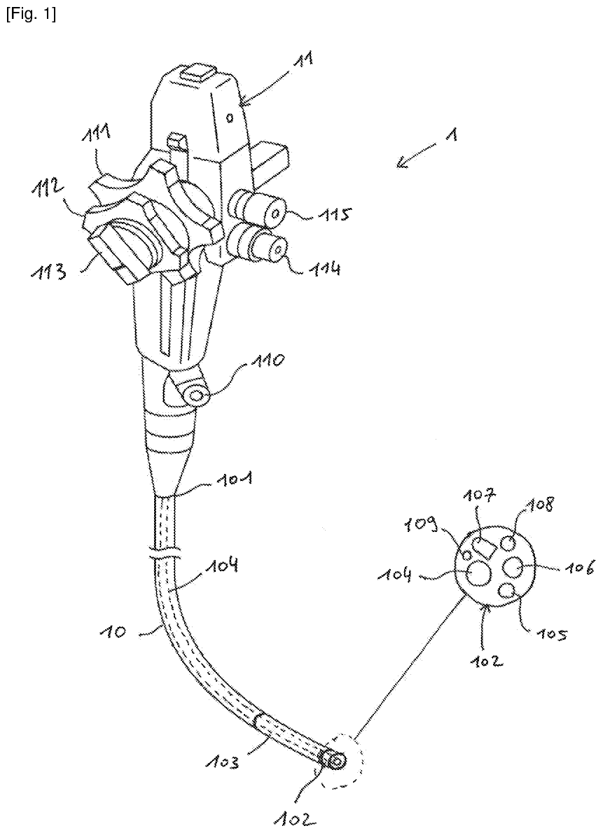

Such a conventional endoscope 1 is shown in FIG. 1. It is a flexible endoscope, also known as an endoscopic probe or fiberscope. It allows the exploration of the internal cavities of the patient's body such as bronchial tubes, esophagus, stomach and others, and surgery on a target zone. It comprises an insertion tube 10 and a control handle 11.

The insertion tube 10 is a flexible tube extending along a longitudinal axis and is intended to be inserted into the patient's body. It is generally cylindrical in shape and formed by a sheath made of a plastic material with an external diameter ranging from 3 mm to 13 mm. The tube 10 has a proximal end 101 integral with the control handle 11 and a distal end 102—or head—which is located in the internal body cavity of the patient when it is in use. The length of the tube 10 between the two ends 101 and 102 ranges from 300 mm to 1500 mm. The head 102 is arranged at the end of a distal portion 103 equipped with tip deflection controlled from the control handle 11.

An operating channel 104 is arranged inside the tube 10. This is a channel that extends longitudinally in the tube 10 and opens at the head 102. This channel 104 makes it possible to insert a surgical instrument (forceps, stapling device, scissors, brush, etc.) into the tube 10 and bring its active portion (or tool) out at the head 102. The surgical instrument is inserted from an entry orifice 110 located at the control handle 11 and which opens into the channel 104. In FIG. 1, there is a single such operating channel 104. In other words, the tube 10 has only one operating channel 104. According to one embodiment, the operating channel 104 can be a double channel, and tube 10 then has two operating channels.

Inside the tube 10, there is also a passage for an optical vision device. That device allows the formation of an image of the intervention area in the cavity. It comprises a light source 105 arranged on the head 102, for example formed by an LED or optical fibers, to illuminate the intervention area. It also includes an image sensor 106 (e.g., CCD camera) arranged on the head 102 to obtain an image of the illuminated intervention area.

Where applicable, the tube 10 can also comprise the following: a passage for injecting water opening at the head 102 through an injection orifice 107; a passage for blowing air opening at the head 102, through a blowing orifice 108; a passage for suctioning fluid opening at the head 102, through a suction orifice 109.

The control handle 11 is designed to be connected to a connector (light, video) via a connecting cord (not shown). In FIG. 1, it has levers for controlling the tip deflection of the distal portion 103. A lever 111 for up/down movement of the tip deflection and a lever 112 for right/left movement of the tip deflection. The cables connecting the levers 111, 112 to the tip deflection pass through the tube 10. When the user operates these levers, they can actuate the deformation and/or angulation of the distal portion 103 and thus of the head 102. They can thus modify its orientation in space. The levers 111, 112 can cooperate with a brake 113 to lock the tip deflection in position. In FIG. 1, the control handle 11 also has a connector 114 for injecting water/air and a connector 115 for fluid suction.

The two endoscopes of the medical device are similar to the one described with reference to FIG. 1. In the description below, the first endoscope is associated with reference “A” and the second endoscope with reference “B”.

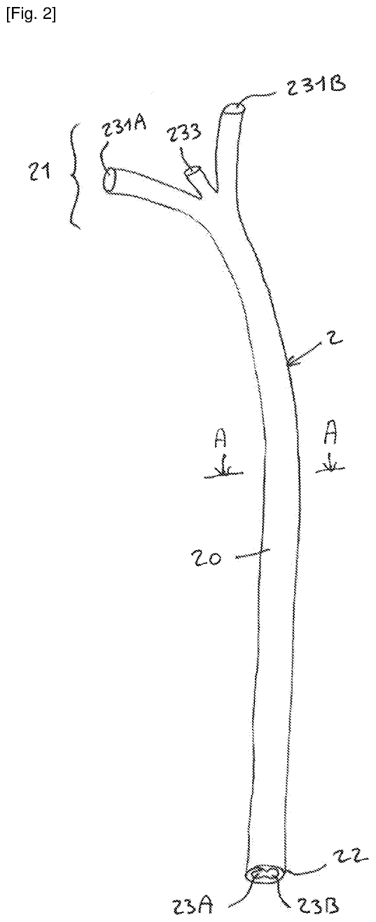

An overtube 2 in accordance with the invention is illustrated in FIGS. 2 to 4. It has a tubular body 20 that is flexible and extends longitudinally. The body 20 is for example made of silicone or polyurethane and obtained by extrusion or molding. It has a proximal portion 21 and an open distal end 22. For example, the length of the body 20 between the two ends 21 and 22 ranges from 250 mm to 1500 mm; its external diameter, or external dimension, ranges from 10 mm to 30 mm; and its thickness from 1 mm to 3 mm.

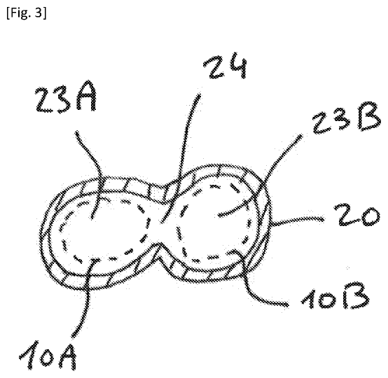

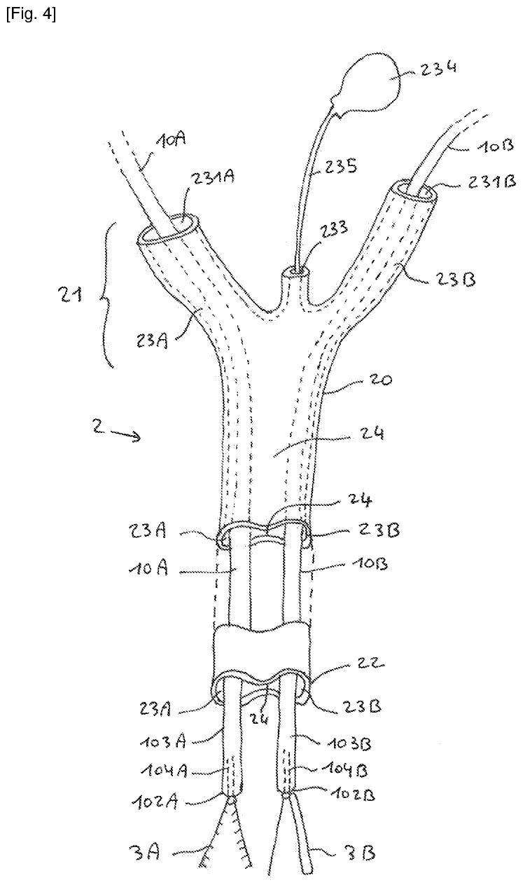

With reference to FIGS. 3 and 4, two ducts 23A, 23B are arranged longitudinally in the body 20. The first duct 23A is designed to accommodate the first insertion tube 10A of the first endoscope 1A. And the second duct 23B is designed to accommodate the second insertion tube 10B of the second endoscope 1B. These two ducts 23A, 23B open at the distal end 22.

The ducts 23A, 23B are configured so that the tubes 10A, 10B can move longitudinally in the body 20. In other words, the tubes 10A, 10B have an up/down translational motion in the body 20. The internal dimensions (diameters) of the ducts 23A, 23B correspond substantially to the external dimensions (diameters) of the tubes 10A, 10B, but are however slightly larger, for example by 0.5 to 1 mm, to facilitate the displacement of said tubes along the longitudinal axis of the body 20. The ducts 23A, 23B also allows rotational movement of the tubes 10A, 10B so that said tubes can rotate on themselves about their longitudinal axis, within their respective duct 23A, 23B.

In FIGS. 2 and 4, the proximal portion 21 of the body 20 is generally W-shaped. It includes a first orifice 231A for introducing the first tube 10A into the first duct 23A and a second orifice 231 B for introducing the second tube 10B into the second duct 23B. A third orifice 233 is also made in the proximal portion 21. This third orifice 233 is used to introduce lubricants into the two ducts 23A, 23B. The lubricant is for example paraffin or glycerine. It reduces friction between the inner wall of the ducts 23A, 23B and the outer wall of the tubes 10A, 10B, so as to facilitate the insertion of said tubes into the body 20 and their longitudinal displacement. The third orifice 233 has, for example, a diameter from 1 mm to 10 mm. The lubricant used may be contained in a flexible reservoir 234 of the bulb or syringe type, connected to the third orifice 233 by a duct 235. By manually applying pressure on the reservoir 234 or syringe, lubricant is dispensed into the body 20.

In FIGS. 3 and 4, the first duct 23A and the second duct 23B communicate with each other, at least along the length of the body 20 that does not include the proximal portion 21, through a communication passage 24. In FIGS. 3 and 4, ducts 23A, 23B are separated and no longer adjacent in the proximal portion 21. According to one embodiment, ducts 23A, 23B are adjacent along their entire length, including in the proximal portion 21, and communicate with each other along their entire length through passage 24. That passage 24 opens at the third orifice 233. Thus, when lubricant is introduced into the body 20 from the third orifice 233, it spreads by gravity simultaneously in both ducts 23A, 23B, along their entire length, so that said ducts are perfectly lubricated.

The first duct 23A, the second duct 23B, and the passage 24 are configured so that the body 20 has an 8-shaped cross section (FIG. 3). That configuration avoids any extra thickness of the wall of the body 20, which not only optimizes its size, but also reduces its manufacturing cost.

The first duct 23A, the second duct 23B and the passage 24 are also configured so that the two tubes 10A and 10B do not touch each other when they are housed in said ducts. That avoids any overlapping and rubbing of the tubes 10A, 10B against each other, which could hinder their insertion and/or movement in the body 20. Preferably, to achieve this technical effect while simplifying the design of the body 20, the passage 24 is narrower than the ducts 23A, 23B.

In FIG. 4, when the insertion tubes 10A, 10B are installed in the body 20 and housed in their respective ducts 23A, 23B, their distal portions 103A, 103B and their heads 102A, 102B protrude out of said ducts and said body, through the distal end 22 of the latter. The distal portions 103A, 103B and the heads 102A, 102B thus protrude from the body 20 so that they are positioned in the body cavity when in use.

In FIG. 4, the instrument 3A inserted into the operating channel 104A of the first tube 10A of the first endoscope 1 A is a pair of forceps, preferably wide-jawed forceps (width from 3 mm to 10 mm), which does not cause trauma. The instrument 3B inserted into the operating channel 104B of the second tube 10B of the second endoscope 1B is a surgical stapling device or mounted clip, allowing the simultaneous placement of one or more staples or clips (e.g., simultaneous placement of multiple rows of staples or clips). The staples or clips used are preferably made of titanium. As an example for guidance, their width is about 3 mm and their length is 3 mm to 5 mm. This type of surgical stapling device is for example marketed by Ethicon Endo Surgery® or Covidien®. When in use, the instruments 3A, 3B emerge from their respective operating channels 104a, 104B, and are located outside the body 20 for positioning in the body cavity.

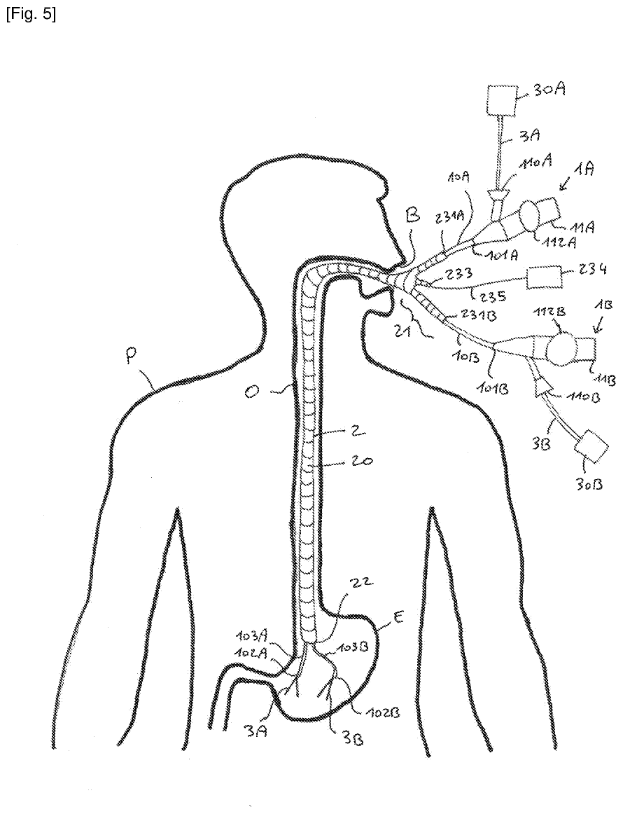

FIG. 5 illustrates the placement of the medical device in the body of a patient P for bariatric surgery. The overtube 2 is first inserted through the mouth B of the patient P, into the esophagus O, until the distal end 22 of the body 20 is located in the stomach E. Such insertion may be performed with guide wire initially placed under gastroscopic control. When the overtube 2 is installed, its proximal portion 21 is located outside the mouth B of the patient P, so that orifices 231A, 231B and 233 are accessible.

The first tube 10A of the first endoscope 1A is then inserted into the body 20. Such insertion is performed from the entry orifice 231A so that the first tube 10 is housed in the first duct 23A. The tube 10A and/or the first duct 23A are advantageously lubricated beforehand. Such insertion is finalized when the distal portion 103A and the head 102A of the first tube 10A emerge from the first duct 23A and are located in the stomach E, close to the intervention area. Such insertion may be performed under the control of the optical vision device of the first endoscope 1A. The second tube 10B of the second endoscope 1B is inserted in the same way.

The first instrument 3A is then inserted into the operating channel 104A of the first tube 10A through the entry orifice 110A. Such insertion is finalized when the tool (e.g., forceps) of the first instrument 3A comes out of the head 102A and is located in the stomach E. In the same way, the second instrument 3B is inserted in the operating channel 104B of the second tube 10B of the second endoscope 1B. The instruments 3A, 3B are operable using control components 30A, 30B located outside the body of the patient P.

If the user needs to move the tools of the instruments 3A, 3B closer to or away from the intervention area, they can manipulate the endoscopes 1A, 1B to slide and/or rotate the tubes 10A, 10B in their respective ducts 23A, 23B. These movements are preferably accompanied by lubrication of the ducts 23A, 23B from the third orifice 233.

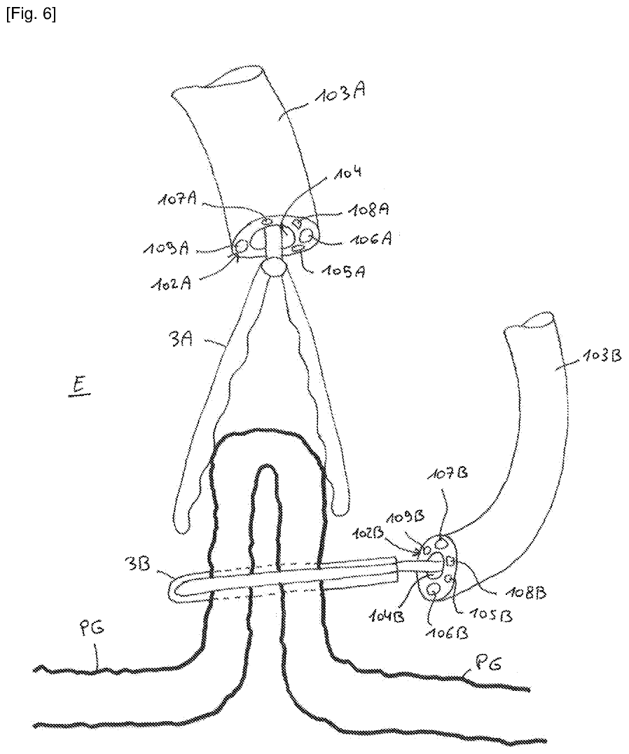

In FIG. 6, the tools of instruments 3A and 3B (forceps and a surgical stapling device respectively) are positioned in the intervention area. Here, that area is a region of the gastric wall PG (peritoneum). The tip deflection of the distal portions 103A, 103B allows each instrument 3A, 3B to be directed specifically; here, the forceps and stapling device are directed at 90°. Each instrument 3A, 3B is additionally viewed by its own camera 106A, 106B. The user therefore has a better view of the intervention area which can be observed from two different angles.

In FIG. 6, the forceps 3A are positioned perpendicular to the wall PG. The forceps are operated to pinch the PG wall and form a wide fold that can carry the peritoneal leaflets. The stapling device or mounted clip 3B is positioned perpendicular to the forceps 34 and thus perpendicular to the fold thus formed. The stapling device or mounted clip 3B is then operated to staple the fold and hold it in position. The PG wall is not stitched, but merely held by clips or staples, which is much faster and less traumatic for the said wall, with no risk of peritoneal breaking.

Furthermore, one or more characteristics outlined only in one embodiment may be combined with one or more other characteristics outlined only in another embodiment.

The arrangement of the different elements and/or means and/or steps of the invention, in the embodiments described above, must not be understood as requiring such an arrangement in all the implementations. Various variants may be used, including:

The part 33 is not necessarily tube-shaped, but may be spherical, ovoid, etc.

The part 33 is not necessarily made of plastic but may also be made of resin, metal, composite, plant material, etc.

1.-12. (canceled)

13. An overtube comprising:
a longitudinally extending flexible tubular body having a proximal portion and an open distal end,
a first duct designed to house a first tube for inserting a first endoscope into the body, with the possibility of longitudinal displacement of said first tube in said first duct and rotational movement of said first tube about its longitudinal axis, which first duct is arranged longitudinally inside said body and opens at the distal end of said body,
a second duct designed to house a second tube for inserting a second endoscope into the body, with the possibility of longitudinal displacement of said second tube in said second duct and rotational movement of said second tube about its longitudinal axis, which second duct is arranged longitudinally inside said body and opens at the distal end of said body,
a first orifice made in the proximal portion of the body for introducing the first insertion tube into the first duct,
a second orifice made in the proximal portion of the body for introducing the second insertion tube into the second duct,
a third orifice made in the proximal portion of the body for introducing lubricant into the first duct and into the second duct,
wherein the first duct and the second duct communicate with each other, at least in the length of the body that does not include the proximal portion, through a communication passage, which opens at the third orifice,
and wherein the communication passage connecting the first duct to the second duct is narrower than said ducts.

14. An overtube according to claim 13, wherein the first duct, the second duct and the communication passage are configured such that the first insertion tube of the first endoscope and the second insertion tube of the second endoscope do not touch each other when said tubes are housed in said ducts.

15. An overtube according to claim 13, wherein the body has an 8-shaped cross section.

16. An overtube according to claim 13, wherein the body has a length of 250 mm to 1500 mm

17. A medical device, comprising:
a first endoscope comprising:
a first flexible insertion tube having a first distal portion equipped with tip deflection, inside which is arranged a first operating channel for inserting a first surgical instrument, which first channel opens at a first distal end of said first tube,
a first optical viewing device located at the first distal end of the first insertion tube,
a first control handle connected to a proximal end of the first insertion tube,
a second endoscope comprising:
a second flexible insertion tube having a second distal portion equipped with tip deflection, inside which is arranged a second operating channel for inserting a second surgical instrument, which second channel opens out at a second distal end of said second tube,
a second optical viewing device located at the second distal end of the second insertion tube,
a second control handle connected to a proximal end of the second insertion tube.

18. An overtube comprising:
a longitudinally extending flexible tubular body having a proximal portion and an open distal end,
a first duct designed to house a first tube for inserting a first endoscope into the body, with the possibility of longitudinal displacement of said first tube in said first duct and rotational movement of said first tube about its longitudinal axis, which first duct is arranged longitudinally inside said body and opens at the distal end of said body,
a second duct designed to house a second tube for inserting a second endoscope into the body, with the possibility of longitudinal displacement of said second tube in said second duct and rotational movement of said second tube about its longitudinal axis, which second duct is arranged longitudinally inside said body and opens at the distal end of said body,
a first orifice made in the proximal portion of the body for introducing the first insertion tube into the first duct,
a second orifice made in the proximal portion of the body for introducing the second insertion tube into the second duct,
a third orifice made in the proximal portion of the body for introducing lubricant into the first duct and into the second duct,
wherein the first duct and the second duct communicate with each other, at least in the length of the body that does not include the proximal portion, through a communication passage, which opens at the third orifice,
and wherein the communication passage connecting the first duct to the second duct is narrower than said ducts, wherein:
the first insertion tube is housed in the first duct, and the first distal portion and the first distal end of said first tube protrude out of said first duct and the body through the distal end of said body,
the second insertion tube is housed in the second duct, and the second distal portion and the second distal end of said second tube protrude out of said second duct and the body through the distal end of said body.

19. A medical device according to claim 17, wherein the first surgical instrument is a pair of wide-jawed forceps.

20. A medical device according to claim 17, wherein the second surgical instrument is a surgical stapling device, or a mounted clip.

21. An overtube according to claim 14, wherein the body has an 8-shaped cross section.

22. An overtube according to claim 14, wherein the body has a length of 250 mm to 1500 mm.

23. An overtube according to claim 15, wherein the body has a length of 250 mm to 1500 mm.

24. A medical device according to claim 18, wherein the second surgical instrument is a surgical stapling device, or a mounted clip.
